(19) United States
(12) Patent Application Publication
(10) Pub. No.: US 2024/0390167 A1
(43) Pub. Date: Nov. 28, 2024

(54) ATTACHMENT SYSTEM FOR PROSTHETIC FOOT ASSEMBLY TO FOOTWEAR

(71) Applicant: Ossur Iceland ehf, Reykjavik (IS)

(72) Inventors: Aron Kristbjorn ALBERTSSON, Reykjavik (IS); Victoria Pamela Anne CLARK, Reykjavik (IS); Felix STARKER, Reykjavik (IS); Markus REHM, Reykjavik (IS); Arinbjorn Viggo CLAUSEN, Reykjavik (IS); Rachel Adsit LOWING, Reykjavik (IS)

(21) Appl. No.: 18/671,061

(22) Filed: May 22, 2024

Related U.S. Application Data
(60) Provisional application No. 63/503,582, filed on May 22, 2023.

(51) Int. Cl. A61F 2/66; A61F 2/50

(52) U.S. Cl. CPC A61F 2/66; A61F 2002/5001; A61F 2002/6614

(57) ABSTRACT

An attachment system for connecting footwear to a prosthetic foot assembly includes a prosthetic foot and a foot cover surrounding the prosthetic foot. The footwear has a footbed defining an insole or inner surface and an outsole or outer surface. The attachment system includes a pin arranged to protrude from the inner surface of the footwear and connect to the prosthetic foot assembly; a mounting plate secured to the pin and arranged to extend along the inner surface of the footwear; at least one fastener arranged to secure the pin to the footbed. A method is provided for using the attachment system.

### INCORPORATION BY REFERENCE TO ANY PRIORITY APPLICATIONS

This application claims priority to U.S. Provisional Application No. 63/503,582, filed May 22, 2023, which is incorporated herein by reference. Any and all applications for which a foreign or domestic priority claim is identified in the Application Data Sheet as filed with the present application are hereby incorporated by reference under 37 CFR 1.57.

### FIELD OF THE DISCLOSURE

The present disclosure relates to an attachment system for connecting footwear to a prosthetic foot assembly and, more specifically, to a prosthetic foot cover or prosthetic foot.

### BACKGROUND

Conventional prosthetic foot designs are sometimes enclosed in a cosmesis or foot cover to improve the aesthetic appeal of the prosthesis. During use, water, dirt, or other debris may become trapped inside the foot cover, requiring the removal of the foot cover to clean the foot cover. Conventional designs have the shape of a human foot and are made, for example, of rubber or other flexible materials. Although these materials are flexible, the foot cover must stay on during use, so foot covers are intentionally difficult to remove. An exemplary foot cover is described in U.S. Nonprovisional application Ser. No. 18/180,757, filed on Mar. 8, 2023, and incorporated herein by reference.

A difficulty arises when an amputee desires to wear certain footwear with a prosthetic foot assembly, including a prosthetic foot and a corresponding cover. In these instances, such as wearing sandals, the prosthetic foot does not connect to a midfoot or heel portion of the footwear, therefore making it challenging to walk with such footwear. In other instances, an attachment system may improve wearing other types of footwear, particularly because the prosthetic foot may not articulate like a sound foot.

Accordingly, there is a need for an attachment system to improve the connection of a prosthetic foot assembly to footwear.

### SUMMARY

Foot covers, also known as cosmeses, are typically manufactured out of one piece of material for prosthetic feet. The material may be flexible, and the foot cover must stay on during use for the foot cover to be useful. While the foot covers approximate and appear as a sound foot, they have limitations in flexibility as the user cannot adapt the foot cover as a normal foot would adapt to footwear. As a result, additional means may be required to maintain the footwear on the foot cover.

For example, if the user wears sandals, such sandals have straps that typically only attach to the toe portion or forefoot of a foot. As a result, a foot cover remains essentially static, and the user cannot control the foot cover like a sound foot. Thus, additional attachment means can be advantageously used to secure the foot cover to the sandal better, aside from a typical strap, to avoid slippage of the sandal. In addition, if an attachment system is provided at the midfoot or heel portion of the footwear and has corresponding components on the foot cover, the footwear can remain more stable on the foot cover.

According to an embodiment, an attachment system connects footwear to a prosthetic foot assembly, including a prosthetic foot and a foot cover surrounding the prosthetic foot. The footwear has a footbed defining an insole or inner surface and an outsole or outer surface. The attachment system includes a pin arranged to protrude from the inner surface of the footwear and connect to the prosthetic foot assembly. A mounting plate is secured to the pin and arranged to extend along the inner surface of the footwear. At least one fastener may be arranged to secure the pin or the mounting plate to the footbed.

The pin may define a head at the end of a shaft extending from a base secured to the mounting plate. However, the pin may take a variety of shapes suitable for selectively engaging the footwear upon donning and use and releasing from the footwear upon doffing.

The mounting plate is preferably fixedly secured to the pin and may form a unitary structure with the pin. However, the mounting plate and the pin may each be formed from different materials or geometrical shapes, leading to different physical properties. For example, the mounting plate may be formed from a plastic molded about and lockingly secured to the pin, which may be formed from metal.

The mounting plate may define at least one wing extending from a center portion of the mounting plate where the pin attaches to the mounting plate. Preferably, the mounting plate includes first and second wings extending oppositely to one another from the center portion and may be commensurately flexible to the footbed to impede or interfere with the user's gait when wearing the footwear.

The mounting plate may be secured to the footwear with an adhesive, or the pin may be fastened to the footbed of the footwear with a fastener. For example, a fastener may engage the pin and extend from the outer surface of the footwear to the inner surface or the pin to secure the pin to the footbed.

In an example of coupling the pin to the foot cover, the foot cover may define an opening configured and dimensioned to receive and engage the pin. The pin may lock onto the foot cover and be maintained during donning and use of the footwear. In addition, other means for securing the pin are envisioned, such as latches, recesses, and other means, including any combination described in this disclosure or those known to one skilled in the art.

In another embodiment for connecting footwear to a prosthetic foot assembly, first and second retainers may be arranged to secure to the footbed. The first and second retainers are preferably arranged to press against an outer surface of the foot cover resiliently. The retainers may be placed at various footwear locations to secure the foot cover, including the medial or lateral sides of the footwear or a posterior or heel portion of the footbed. In addition, the retainers may be adhered to or inserted into a thickness of the footbed to secure and maintain hold of the foot cover.

In yet another embodiment for connecting footwear to a prosthetic foot assembly, the attachment system includes a first locking part located along the footbed and a second locking part located along the outsole of the foot cover, such that the second locking part corresponding to the first locking part to interlocks with the second locking part.

Various means may be used to form the first and lock the parts. For example, the first locking part may define a pin arranged to lock mechanically with the foot cover at an opening. The second locking part may define a receptacle adapted to receive the pin. The first and second locking parts have magnetic elements arranged to lock together magnetically. The first locking part may be embedded in the footbed, and the pin protrudes. The second locking part may be embedded in the outer surface of the foot cover.

The attachment system is particularly adapted for sandals in yet another embodiment for connecting footwear to a prosthetic foot assembly. A toe block may be arranged to secure the footbed of the footwear. The toe block defines a toe contour adapted to receive the corresponding contours of the foot cover so that a toe portion of the foot cover rests and engages the toe block. The toe block may also define a forefoot contour adapted to receive the corresponding contours of the foot cover. The toe block may have a toe separator defined within the toe contour and corresponds to a toe split of the foot cover. The toe block may define a block base adjacent to the footbed and may adhere to the insole of the footwear with an adhesive.

While the embodiments are described with a foot cover surrounding a prosthetic foot, they may be adapted to secure footwear directly to the prosthetic foot. Likewise, the embodiments are not limited to securing footwear from sandals to a prosthetic foot assembly but may be adapted to secure other footwear, such as shoes.

Numerous other advantages, features, and functions of embodiments of an attachment system will become readily apparent and better understood because of the following description and accompanying drawings.

### GLOSSARY

Conditional language, such as “can,” or “may,” unless specifically stated otherwise, or otherwise understood within the context as used, is generally intended to convey that certain embodiments include, while other embodiments do not include, certain features or elements.

Conjunctive language such as “at least one of [various elements],” unless specifically stated otherwise, is otherwise understood with the context used in general to convey that an item, term, etc., may be any of the elements listed. Thus, such conjunctive language is not generally intended to imply that certain embodiments require the presence of at least one of each element.

Language of degree used herein, such as the terms “approximately,” “about,” “generally,” and “substantially” as used herein, represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5degrees, 3 degrees, 1 degree, 0.1 degrees, or otherwise. Additionally, as used herein, “gradually” has its ordinary meaning (e.g., it differs from a non-continuous, such as a step-like change).

Footwear can be understood as any item worn on the foot. Sandals are described here as exemplary footwear, but such an example is not meant to limit the scope of the disclosure.

Fasteners can be made of a clasp, snap, magnet, button, hook-and-loop, elastic band, hook, or any other viable element known to one skilled in the art.

Inner or interior can mean the portion of the footwear closest to the prosthetic.

Outer or exterior can mean the footwear portion furthest from the prosthetic or be considered the opposite of inner.

### BRIEF DESCRIPTION OF THE DRAWINGS

The drawing figures are not necessarily drawn to scale but instead are drawn to provide a better understanding of the components thereof. They are not intended to be limiting in scope but to provide exemplary illustrations. The figures illustrate exemplary configurations of an orthopedic device and in no way limit the structures or configurations of an attachment system for a prosthetic foot assembly according to the present disclosure.

FIG. 1 is a perspective view of a first attachment system securing footwear to a prosthetic foot assembly.

FIG. 2 is a perspective view showing the donning or doffing of the first attachment system in FIG. 1 to a prosthetic foot assembly and footwear.

FIG. 3 is a perspective view showing the footwear's insole or inner surface, including components of the first attachment system.

FIG. 4 is a perspective view of the pin and mounting plate in the first attachment system of FIG. 1.

FIG. 5 is a schematic view of the pin and a fastener in the first attachment system.

FIG. 6 is a top sectional plan view showing the interior of a foot cover adapted for forming an opening in the first attachment system.

FIG. 7 is a bottom sectional plan view showing a preform defined by the foot cover on the exterior of the foot cover for forming an opening in the first attachment system.

FIG. 8 is a bottom plan view of the foot cover, including the opening formed according to the interior and exterior means of the foot cover in FIGS. 6 and 7.

FIG. 9 is a top plan view showing a second attachment system for a prosthetic foot assembly to footwear.

FIG. 10 is a perspective view of the second attachment system of FIG. 9.

FIG. 11 is an elevational view showing the second attachment of FIG. 9.

FIG. 12 is an exemplary perspective view showing a third attachment system of a prosthetic foot assembly to footwear.

FIG. 13 is a bottom sectional plan view showing an outsole or outer surface of the foot cover having a second locking part in the third attachment system of FIG. 11.

FIG. 14 is a perspective view showing a fourth attachment system of a prosthetic foot assembly to footwear.

FIG. 15 is a perspective view showing the fourth attachment system of FIG. 14 with the foot cover removed from the footwear.

FIG. 16 is a perspective view showing a toe block in the fourth attachment system of FIG. 14.

FIG. 17 is an exemplary view showing retention of the toe portion or forefoot of the foot cover by the toe block.

FIG. 18 is a perspective view of the toe block, toe contour, forefoot contour, and toe block base in the fourth attachment system of FIG. 14.

### DETAILED DESCRIPTION OF VARIOUS EMBODIMENTS

### A. Overview

A better understanding of different embodiments of the disclosure may be had from the following description read with the accompanying drawings in which reference characters refer to like elements.

While the disclosure is susceptible to various modifications and alternative constructions, certain illustrative embodiments are in the drawings and are described below. It should be understood, however, that there is no intention to limit the disclosure to the specific embodiments disclosed. On the contrary, the intent is to cover all modifications, alternative constructions, combinations, and equivalents falling within the spirit and scope of the disclosure.

### B. Various Embodiments of Attachment Systems for Connecting a Prosthetic Foot Assembly to Footwear

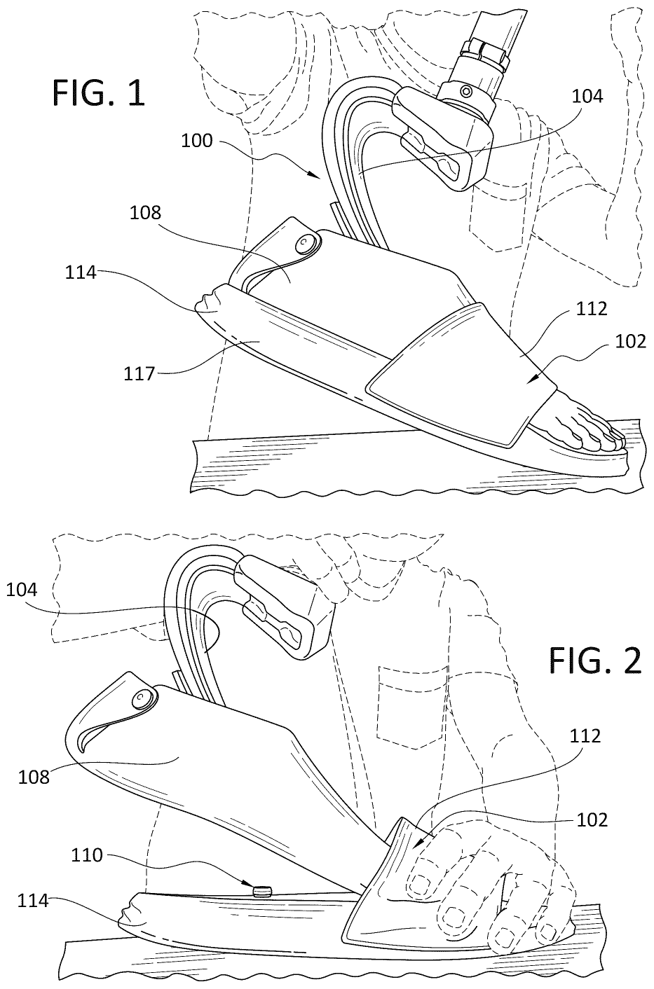

In the first embodiment, FIGS. 1 and 2 depict an attachment system 110 for connecting footwear 102 to a prosthetic foot assembly 100, including a prosthetic foot 104 and a foot cover 108 surrounding the prosthetic foot 104. An exemplary prosthetic foot is found in U.S. Patent Nos. 9,017,42 granted Apr. 28, 2015, and 11,147,692, granted Oct. 19, 2021; and an exemplary foot cover is found in U.S. Design Patent No. D795,433, granted Aug. 22, 2017; each of these exemplary patents is incorporated herein by reference. For reference, the footwear 102 has a footbed 114 defining an insole or inner surface 116 and an outsole or outer surface 117. While the footwear is depicted as a sandal, a variety of footwear may be adapted with the attachment system to improve the connection of a prosthetic foot assembly to a footwear.

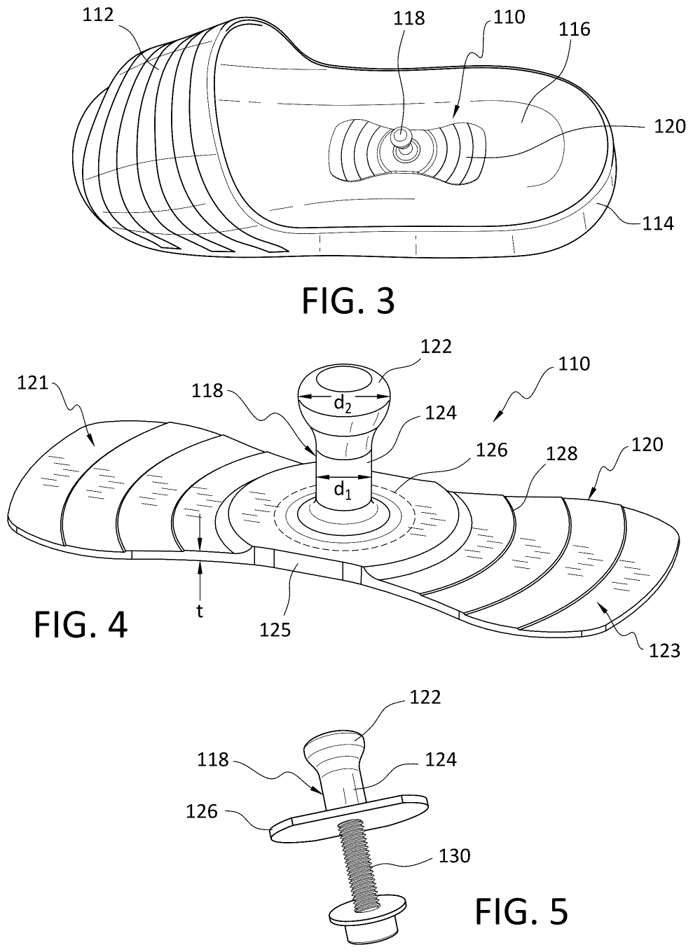

FIGS. 3-5 show an exemplary pin 118 according to the first embodiment. The pin 118 is arranged to protrude from the inner surface 116 of the footwear 102 and connect to the prosthetic foot assembly 100. A mounting plate 120 may be secured to the pin 118 and arranged to extend along the inner surface 116 of the footwear 102. At least one fastener 130 may be arranged to secure the pin 118 to the footbed 114.

For example, the pin 118 may define a head 122 at the end of a shaft 124 extending from a base 126 secured to the mounting plate 120. The head 122 may preferably have a greater diameter d2 than a diameter d1 of the shaft 124 to improve interlocking with an opening defined by the foot cover 108. While the pin 118 is described and illustrated, such is only an example, and a variety of configurations of a pin or similar component may be used to connect to a corresponding feature defined by the foot cover 108.

FIGS. 3 and 4 illustrate an exemplary mounting plate 120 that may be more flexible than the pin 118 and is not required with the pin 118 yet offers greater stability and distribution of the pin 118 against the footbed 114. The mounting plate 120 may be fixedly secured to the base 126 of the pin 118, and flexed against the footbed 114 to not impede the function and performance of the footwear 102. The base 126 may be embedded in a thickness t of the center portion 125, and the shaft 124 protrudes from the mounting plate 120. The mounting plate 120 and pin 118 may be formed from various materials; however, in a preferred embodiment, the mounting plate 120 is formed from plastic, and the pin 118 is formed from metal. Generally, however, the mounting plate 120 is preferably flexible, and the pin 118 is rigid to withstand and maintain a connection with the prosthetic foot assembly 100.

As shown in FIGS. 3 and 4, the mounting plate 120 includes at least one wing 121, 123 extending from a center portion 125 of the mounting plate 120, whereas the base 126 of the pin 118 attaches to the mounting plate 120. The at least one wing 121, 123 is commensurately flexible to the footbed 114. The at least one wing 121, 123 may be rotatable relative to the footbed 114, as well as the pin 118, although the pin 118 may be sufficiently secured to the footbed 114 to resist rotation. A length of the at least one wing 121, 123 may be positioned to align with a longitudinal axis of the footbed 114.

A thickness t of the at least one wing 121, 123 tapers 128 from the center portion 125 to an end portion of the at least one wing 121, 123. The at least one wing 121, 123 may secure to the inner surface 116 of the footbed 114 with an adhesive or may be free from adhesive and frictionally engages the footbed 114.

FIG. 5 illustrates a fastener 130 arranged to engage the pin 118. For example, the pin 118 may define a central threaded bore (not shown) to engage the fastener 130. The fastener 130 preferably defines a length greater than the thickness of the footbed 114 such that the at least one fastener 130 engages the outer surface 117 of the footbed 114 and extends therethrough to engage the shaft 124.

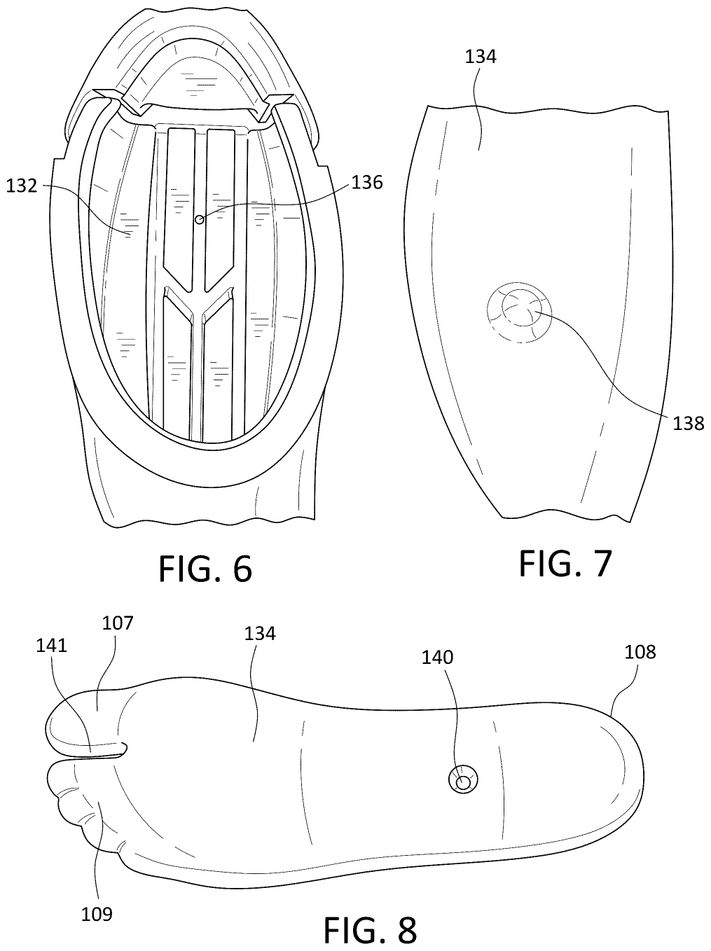

FIG. 6 shows that the foot cover 108 may define an aperture 136 configured and dimensioned to receive and engage the head 122 of the pin 118 for securing therewith, or for being expanded to accommodate the pin 118 for securing therewith.

FIGS. 7 and 8 illustrate the foot cover 108 as having an outer surface 134 defining a preform recess 138. An inner surface 132 defining an aperture 136 corresponds to the preform recess 138, such that puncturing of the preform recess 138 forms an opening 140 adapted to receive the pin 118.

FIG. 8 shows that the foot cover 108 may define a toe split 141 forming a channel adapted to receive a toe anchor 103 of footwear 102.

A method is provided for attaching footwear 102 to a prosthetic foot assembly 100 including the prosthetic foot 104 and a foot cover 108 arranged for surrounding the prosthetic foot 104. The method includes attaching the attachment system 110 to the footwear 102. The attachment system 110 may include the pin 118 protruding from the footwear 102. The pin 118 may engage the foot cover 108 to secure the footwear 102 to the prosthetic foot assembly 100. If the footwear 102 is a sandal, the cover 108 may have a toe opening 105, 141 separating a first toe portion 107 from a second toe portion 109 of the cover 108. The cover 108 may engage a toe anchor 103 by the toe opening 105. The method may include slipping a strap 112 of the footwear 102 over the first and second toe portions 107, 109.

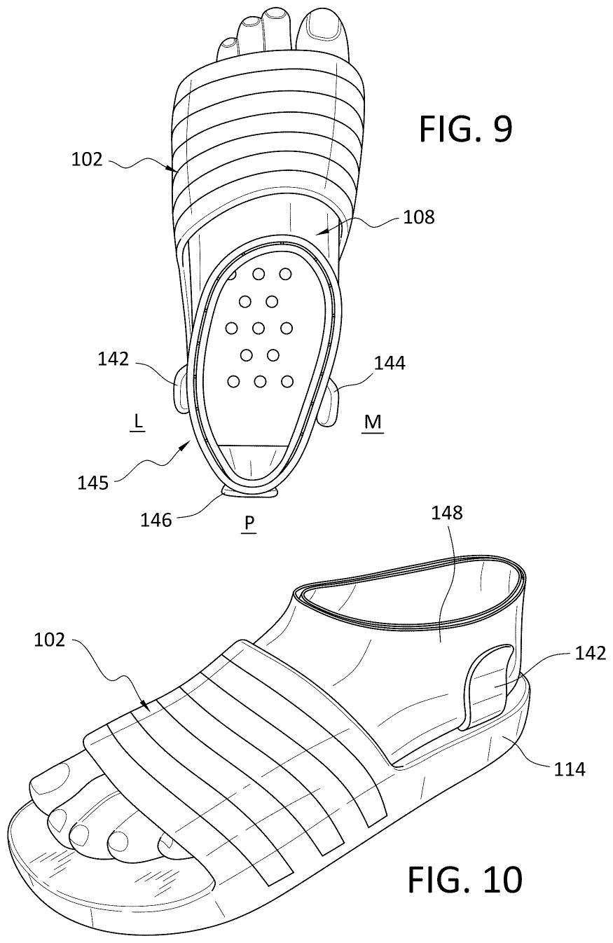

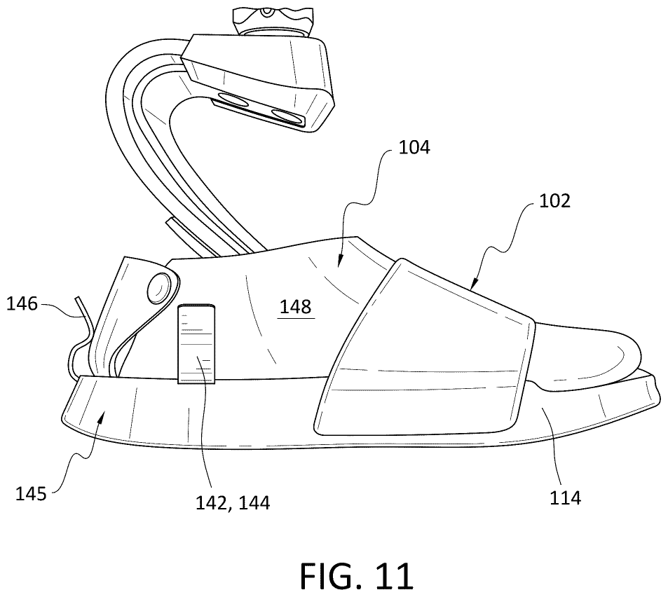

In another embodiment illustrated in FIGS. 9-11, an attachment system 145 includes first and second retainers 142, 144 and a posterior retainer 146 arranged to secure to the footbed 114. The first and second retainers 142, 144 are preferably arranged to press resiliently against an outer surface 148 of the foot cover 108. The retainers 142, 144, 146 may be arranged about the footwear, such as along lateral L, medial M and posterior or heel sides P.

The retainers 142, 144 may be formed as an elongated and flat spring extending over a posterior side P of the heel portion foot cover 108. Of course, other springs or clamps are envisioned that can clamp or press against the cover to secure the footwear to the foot cover.

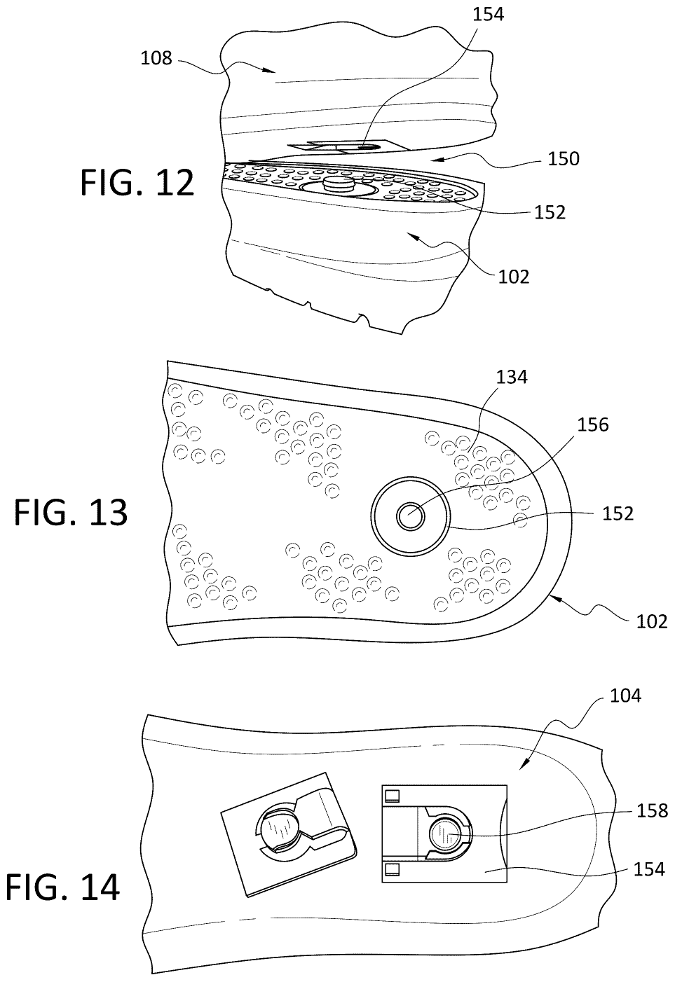

FIGS. 12-14 depict another embodiment of an attachment system 150. The attachment system 150 includes a first locking part 152 located along the footbed 114. A second locking part 154 is located along the outsole 117. The second locking part 154 corresponds to the first locking part 152 to interlock therewith. The first locking part 152 preferably defines a pin 156 arranged to mechanically lock with a receptacle of the second locking part 154 adapted to receive the pin 156.

The first locking part 152 and the second locking part 154 preferably have magnetic elements arranged to lock together magnetically. The first locking part 152 is embedded in the footbed 114 and the pin 156 protrudes therefrom. The second locking part 154 is embedded in the outer surface 107 of the foot cover 108. The second locking part 154 defines an opening 158 configured and dimensioned to receive and engage the pin 156.

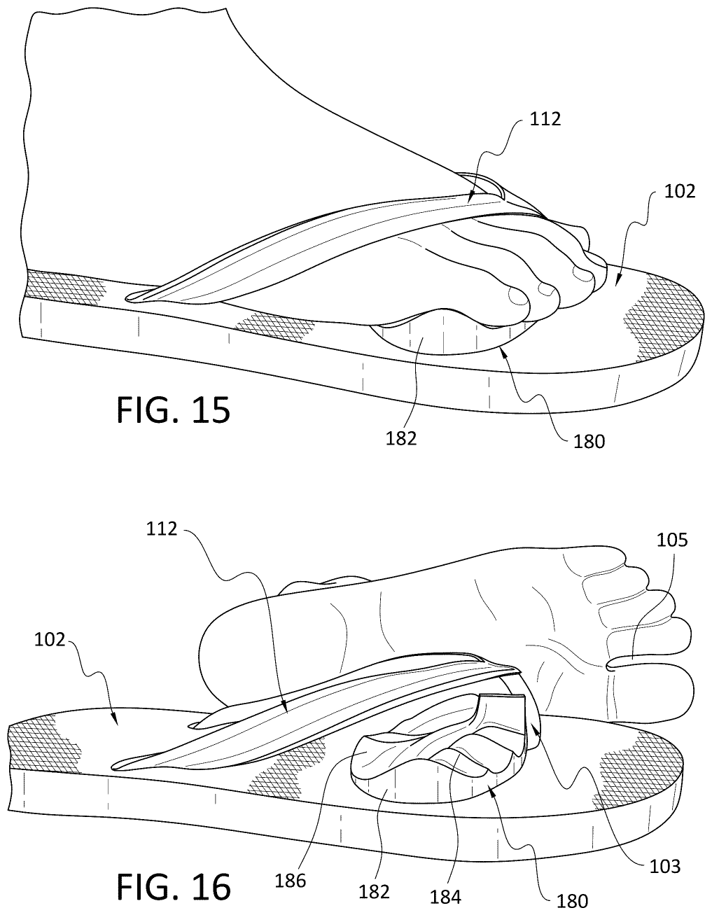

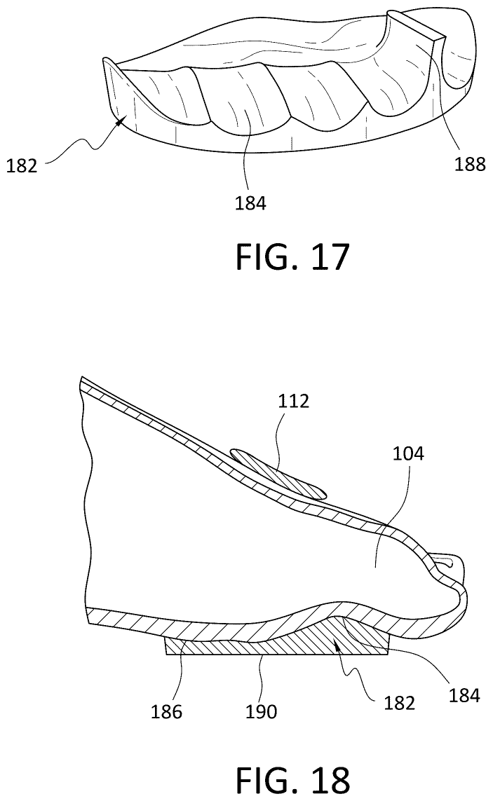

FIGS. 15-18 exemplify yet another embodiment of an attachment system 180. The attachment system 180 includes a toe block 182 arranged to secure to the footbed 114 of the footwear 102. The toe block 182 defines a toe contour 184 adapted to receive corresponding contours of the foot cover 108. The toe block 182 may define a forefoot contour 186 adapted to receive corresponding contours of the foot cover 108. The toe block 182 may define a toe separator 188 defined within the toe contour 184. The toe block 182 defines a toe block base 190 adjacent to the footbed 114 and may be adhered to the footbed 114.

The toe block 182 may be configured as a pin or an element having a shaft carrying the toe block 182. As with any of the aforementioned embodiments, the toe block 182 may have a surface texture that can better engage the foot cover by enhancing frictional properties.

It is understood that not all objects or advantages may be achieved under an embodiment of the disclosure. For example, those skilled in the art will recognize that an attachment system for a prosthetic foot assembly to footwear may be embodied or carried out, so it achieves or optimizes one advantage or group of advantages as taught herein without achieving other objects or advantages as taught or suggested herein.

The skilled artisan will recognize the interchangeability of various disclosed features, including materials, characteristics, and groups. Besides the variations described, other known equivalents for each feature can be mixed and matched by one of ordinary skill in this art to build and use an attachment system under the principles of the present disclosure. Furthermore, the skilled artisan will understand that the features described may be adapted to other methods and types of orthopedic or prosthetic devices.

Although this disclosure describes certain exemplary embodiments and examples of an attachment system, it will be understood by those skilled in the art that the present disclosure extends beyond the specifically disclosed components to other alternative embodiments and/or uses of the disclosure and obvious modifications and equivalents thereof. It is intended that the present disclosure should not be limited by the disclosed embodiments described above and may be extended to other applications that may employ the features described.

1. An attachment system for connecting footwear to a prosthetic foot assembly including a prosthetic foot and a foot cover surrounding the prosthetic foot, the footwear having a footbed defining an insole or inner surface and an outsole or outer surface, the attachment system comprising:
a pin arranged to protrude from the inner surface of the footwear and connect to the prosthetic foot assembly;
a mounting plate secured to the pin and arranged to extend along the inner surface of the footwear;
at least one fastener arranged to secure the pin to the footbed.

2. The attachment system of claim 1, wherein the pin defines a head at an end of a shaft extending from a base secured to the mounting plate, the head having a greater diameter than a diameter of the shaft.

3. The attachment system of claim 2, wherein the mounting plate is more flexible than the pin.

4. The attachment system of claim 2, wherein the mounting plate is fixedly secured to the base of the pin.

5. The attachment system of claim 2, wherein the base is embedded in a thickness of the center portion, and the shaft protrudes from the mounting plate.

6. The attachment system of claim 1, wherein the mounting plate is formed from plastic and the pin is formed from metal.

7. The attachment system of claim 2, wherein the mounting plate includes at least one wing extending from a center portion of the mounting plate whereat the base of the pin attaches to the mounting plate.

8. The attachment system of claim 7, wherein the at least one wing is commensurately flexible to the footbed.

9. The attachment system of claim 7, wherein the at least one wing is rotatable relative to the footbed.

10. The attachment system of claim 7, wherein a length of the at least one wing aligns with a longitudinal axis of the footbed.

11. The attachment system of claim 7, wherein a thickness of the at least one wing tapers from the center portion to an end portion of the at least one wing.

12. The attachment system of claim 7, wherein the at least one wing secures to the inner surface of the footbed.

13. The attachment system of claim 2, wherein the shaft defines a central threaded bore arranged to engage the at least one fastener, the at least one fastener defining a length greater than a thickness of the footbed such that the at least one fastener engages the outer surface of the footbed and extends therethrough to engage the shaft.

14. The attachment system of claim 2, wherein the foot cover defines an opening configured and dimensioned to receive and engage the head of the pin for securing therewith.

15. The attachment system of claim 14, wherein the foot cover includes outer surface defining a preform recess, and an inner surface defining an aperture corresponding to the preform recess, wherein puncturing of the preform recess forms an opening adapted to receive the pin.

16. A method for attaching footwear to a prosthetic foot assembly including a prosthetic foot and a cover surrounding the prosthetic foot, the method comprising the steps of:
attaching an attachment system to the footwear, the attachment system including a pin protruding from the footwear;
engaging the pin to the cover to secure the footwear to the prosthetic foot assembly.

17. The method of claim 16, further comprises providing the cover with a toe opening separating a first toe portion from a second toe portion of the cover; and
engaging a toe anchor to the toe opening.

18. An attachment system for connecting footwear to a prosthetic foot assembly including a prosthetic foot and a foot cover surrounding the prosthetic foot, the footwear having a footbed defining an insole or inner surface and an outsole or outer surface, the attachment system comprising:
first and second retainers and a posterior retainer arranged to secure to the footbed, the first and second retainers arranged to resiliently press against an outer surface of the foot cover.

19. The attachment system of claim 18, wherein at least one of the first and second retainers extends from a heel portion of the footbed.

20. The attachment system of claim 19, wherein the at least one of the first and second retainers is an elongate and flat spring extending over at least one of a medial or lateral side of the heel portion foot cover.
